(19) United States
(12) Patent Application Publication
(10) Pub. No.: US 2007/0129666 A1
(43) Pub. Date: Jun. 7, 2007

(54) System and method of modular integration of intravascular gas exchange catheter with respiratory monitor and ventilator

(76) Inventors: David F. Barton, McFarland, WI (US); Robert Q. Tham, Middleton, WI (US)

Correspondence Address: ANDRUS, SCEALES, STARKE & SAWALL, LLP, 100 EAST WISCONSIN AVENUE, SUITE 1100, MILWAUKEE, WI, 53202, US

(21) Appl. No.: 11/284,691

(22) Filed: Nov. 22, 2005

(51) Int. Cl. A61M 37/00; A61M 31/00

(52) U.S. Cl. 604/26; 600/532; 604/508

(57) ABSTRACT

The present invention is a system and method of integrating an intravascular gas exchange catheter with a patient respiratory system including a monitor and ventilator. The system and method obtains a monitoring sample of respiratory mechanic parameters for a present time interval, which may be selectively recurring over a predefined time. The system and method, according to the aforementioned respiratory mechanic parameters, alerts a physician to adjust, or automatically adjusts the oxygen delivery through the IGEC the ventilator operation, or both the IGEC and ventilator.

### FIELD OF THE INVENTION

The present invention relates to the field of patient ventilation. More specifically, the present invention relates to the field of artificial lung assist devices.

### BACKGROUND OF THE INVENTION

During intensive care therapy for patients with Chronic Obstructive Pulmonary Disease (COPD) and Acute Respiratory Distress Syndrome (ADRS), it is common for clinicians to utilize a respiratory carestation consisting of a critical care ventilator, a respiratory monitor and an information management system. The ventilator provides for the work of breathing based on the patient's clinical needs. The respiratory monitor allows the clinician to view patient waveforms, trends, gas monitoring including inspired and expired O2 and CO2 concentrations, End Tidal CO2 (ETCO2), CO2 production and O2 consumption, metabolics and energy expenditure, as well as patient spirometry. The information management system provides for patient data to be evaluated by the clinician either at the bedside or at a remote location.

An Intravascular Gas Exchange Catheter (IGEC), which in effect is an artificial lung assist device, consists of a multi-lumen catheter with a cylindrical bundle of microporous hollow fiber membranes woven into a mat at the end. The catheter is placed within the central venous blood stream in the primary vein that returns blood to the heart. Once inserted, oxygen gas flows from outside the patient, through the catheter and through the hollow fibers. As blood passes over the fibers, oxygen diffuses into the blood stream from the fibers, while carbon dioxide diffuses out of the blood stream into the fibers. Excess O2 and CO2 are removed back through the catheter out of the body. The device is inserted percutaneously via the femoral vein. A sutureless securement system with anti-microbial agents is then used to hold the catheter in place. The catheter fibers and components are coated with heparin to prevent coagulation.

Operation of Intravascular Gas Exchange Catheters has been discussed in prior-art literature. In particular, in U.S. Pat. No. 4,850,958 (apparatus for extra-pulmonary blood gas exchange) and U.S. Pat. No. 5,219,326 (inflatable percutaneous oxygenator). In other words, prior-art IGEC systems are essentially stand-alone devices that are controlled by an oxygenator.

### SUMMARY OF THE INVENTION

The present invention is a system and method of integrating an intravascular gas exchange catheter with a patient respiratory system including a monitor and ventilator. The system and method obtains a monitoring sample of respiratory mechanic parameters for a present time interval, which may be selectively recurring over a predefined time. The system and method, according to the aforementioned respiratory mechanic parameters, alerts a physician to adjust, or automatically adjusts the oxygen delivery through the IGEC, the ventilator operation, or both the IGEC and the ventilator.

A method of providing integrated care to a patient with an intravascular gas exchange catheter (IGEC) and a carestation comprising collecting a monitoring sample of respiratory parameters with the carestation, determining whether the monitoring sample is within a predefined acceptable range, and adjusting the IGEC when the monitoring sample is not within the predetermined range, wherein adjusting the IGEC controls an amount of oxygen that is added to the bloodstream of the patient and an amount of carbon dioxide removed from the bloodstream of the patient. The method further comprising activating an alarm means when the monitoring sample is not within the predetermined range, wherein the adjusting step is effectuated manually by a user and further comprising coupling the carestation with the IGEC, wherein the adjusting step is effectuated automatically when the carestation sends an instruction signal to the IGEC, and further comprising adjusting a ventilator in combination with the adjusting of the IGEC when the monitoring sample is not within the predetermined range. The collecting step is periodically activated when a user sets the carestation to an auto setting, and the method further comprising setting the IGEC to a starting level based on a set of patient physiological data and setting the predetermined acceptable range on the carestation based on a set of patient physiological data, wherein the IGEC is inserted into the patient through the femoral vein and wherein the carestation includes a critical care ventilator, a respiratory monitor and an information management system. The method, wherein the carestation is configured to monitor any of the following respiratory parameters: inspired and expired O2 and CO2 concentrations, end tidal CO2, CO2 production, O2 consumption, metabolics and energy expenditure, and patient spirometry.

A system of providing integrated care to a patient comprising a carestation configured to collect a monitoring sample of respiratory parameters, wherein the carestation determines whether the monitoring sample is within a predefined acceptable range, and an intravascular gas exchange catheter (IGEC) coupled to the carestation and inserted into the bloodstream of the patient, wherein the IGEC is adjustable when the monitoring sample is not within the predetermined range, wherein adjusting the IGEC controls an amount of oxygen that is added to the bloodstream of the patient and an amount of carbon dioxide removed from the bloodstream of the patient. The system further comprising an alarm means, wherein the alarm means is activated when the monitoring sample is not within the predetermined range, wherein the IGEC is adjusted manually by a user, and wherein the IGEC is adjusted automatically when the carestation sends an instruction signal to the IGEC, wherein the carestation includes a critical care ventilator, further wherein the critical care ventilator is adjustable when the monitoring sample is not within the predetermined range. The system, wherein the carestation collects the monitoring sample periodically when a user sets the carestation to an auto setting, wherein the IGEC is set to a starting level based on a set of patient physiological data, wherein the carestation is set to the predetermined acceptable range based on a set of patient physiological data, wherein the IGEC is inserted into the patient through the femoral vein, and wherein the carestation includes a critical care ventilator, a respiratory monitor and an information management system. The system, wherein the carestation is configured to monitor any of the following respiratory parameters: inspired and expired O2 and CO2 concentrations, end tidal CO2, CO2 production, O2 consumption, metabolics and energy expenditure, and patient spirometry.

A method of providing integrated care to a patient with an intravascular gas exchange catheter (IGEC) and a carestation comprising coupling the carestation with the IGEC, collecting a monitoring sample of respiratory parameters with the carestation, wherein the carestation includes a critical care ventilator, a respiratory monitor and an information management system, determining whether the monitoring sample is within a predefined acceptable range, activating an alarm means when the monitoring sample is not within the predetermined range, and adjusting the IGEC and the critical care ventilator when the monitoring sample is not within the predetermined range, wherein adjusting the IGEC controls an amount of oxygen that is added to the bloodstream of the patient and an amount of carbon dioxide removed from the bloodstream of the patient.

### BRIEF DESCRIPTION OF THE DRAWINGS

FIG. 1 illustrates a block diagram according to an embodiment of the present invention.

FIG. 2 illustrates a flow chart depicting a method of an embodiment of the present invention.

### DETAILED DESCRIPTION OF THE INVENTION

The prior-art IGEC literature describes methods to operate an IGEC as a stand-alone device. However, the prior art does not describe the use of the IGEC in conjunction with a respiratory carestation including a ventilator, a respiratory monitor and an information management system. If used in conjunction with a respiratory carestation, the IGEC could provide the benefits of O2 and CO2 gas exchange while the ventilator is set on less aggressive settings. Significant side benefits for patients include reduced ventilator induced lung damage. It would also reduce the length of time to wean the critical care patient off the ventilator. The conjunction of the IGEC with the respiratory carestation could also significantly decrease the patient's ICU length of stay which improves patient quality of life and reduces cost to the healthcare system.

Recently, point of care therapy delivery devices (e.g. ICU ventilators) have further evolved to integrate more monitoring and therapy functions as well as serve as a bi-directional portal for the broader patient information network. These carestations integrate the activities of a variety of functions in using a common user interface and ergonomic physical function. On certain models, ventilation therapy is integrated with drug delivery through nebulizers, respiratory parameter measurements, respiratory gas monitoring, spirometry and metabolic monitoring. Similar carestations will integrate other types of physiologic monitoring as well, such as ECG, pulse oximetry and entropy. In addition, broadband communication capabilities to obtain information from electronic patient records such as pharmacy and lab data, for example blood gases, and digital imaging information.

One of the advantages of the carestation approach is that it allows the monitoring of therapy devices such as the IGEC so that the therapeutic benefits of the IGEC can be intimately linked to that of other therapy, for example ventilators and monitoring, patient gas monitoring, lung mechanics monitoring, and enhanced by the higher level of information present on the carestation. By integrating the monitoring of the IGEC in conjunction with this information, improved patient outcome can be obtained, especially for patients suffering from COPD and ARDS.

Specifically, the present invention relates to a respiratory therapy carestation, defined as the combination of at least a ventilation delivery device (ventilator) and IGEC. The carestation has a fundamental ability to evaluate the oxygenation level of a patient's blood and provides the clinician with that information during the time that the patient is mechanically ventilated. With this ability the carestation is claimed to provide optimization of oxygen delivery and carbon dioxide removal.

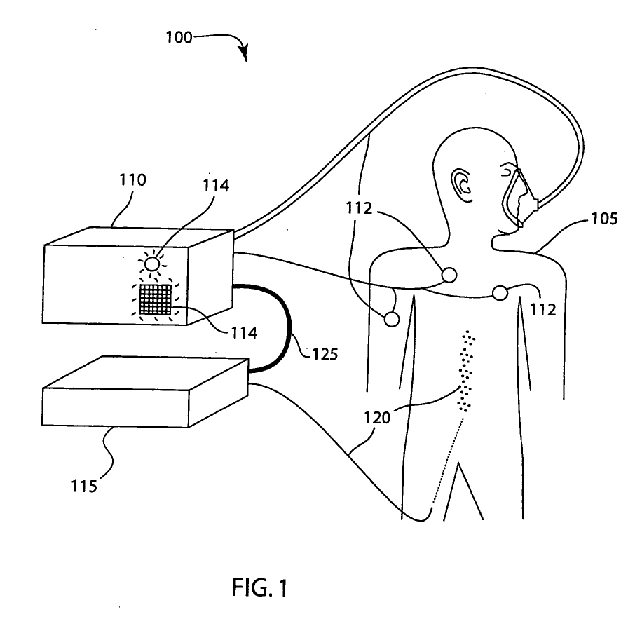

The respiratory carestation system 100 of the present invention is depicted in FIG. 1. In FIG. 1, a patient 105 is monitored by a carestation 1 10, utilizing a number of physiological sensors 112, as required to collect the various physiological parameters set as patient waveforms, trends, gas monitoring, including inspired and expired O2 and CO2 concentrations, end title CO2 (ETCO2), CO2 production and O2 consumption, metabolic and energy expenditure, as well as patient spirometry. The carestation 110 collects this information from the patient 105 and compares it to an acceptable predetermined and preset range. If the physiological parameters of the patient 105 are not within that predetermined, preset range, the alarming means 114 of the carestation 112 will alert a user of the respiratory carestation system 100 of such a condition. The alarming means 114 may be visual, such as a light, and/or an audible alarm. The alarming means 114 will alert a user of the respiratory carestation system 100, so that the user may adjust the IGEC control 115 accordingly, so that the patient 105 may receive the appropriate amount of blood oxygenation from the IGEC 120. The respiratory system 100 is also configured such that the user may adjust the ventilator in the carestation 110, or a combination of the ventilator and the IGEC 120 in order to return the patient's 105 physiological parameters to the acceptable range.

Still referring to FIG. 1, the IGEC 120 is preferably inserted through the femoral vein of the patient 105, and operates as described above. The IGEC 120 is controlled by an IGEC control 115, and is coupled through an IGEC coupling 125 to the carestation 110. In additional embodiments of the present invention, when the patient 105 is displaying parameters that are outside the predetermined, preset range, the carestation 110 will detect this condition, and instruct the IGEC control 115 through the IGEC coupling 125 to adjust the oxygenation through the IGEC 120 automatically, and as described previously, the system 100 will be configured to adjust the ventilator automatically, as well as the ventilator and IGEC 120 in combination in order to return the patients 105 physiological parameters back to an acceptable range.

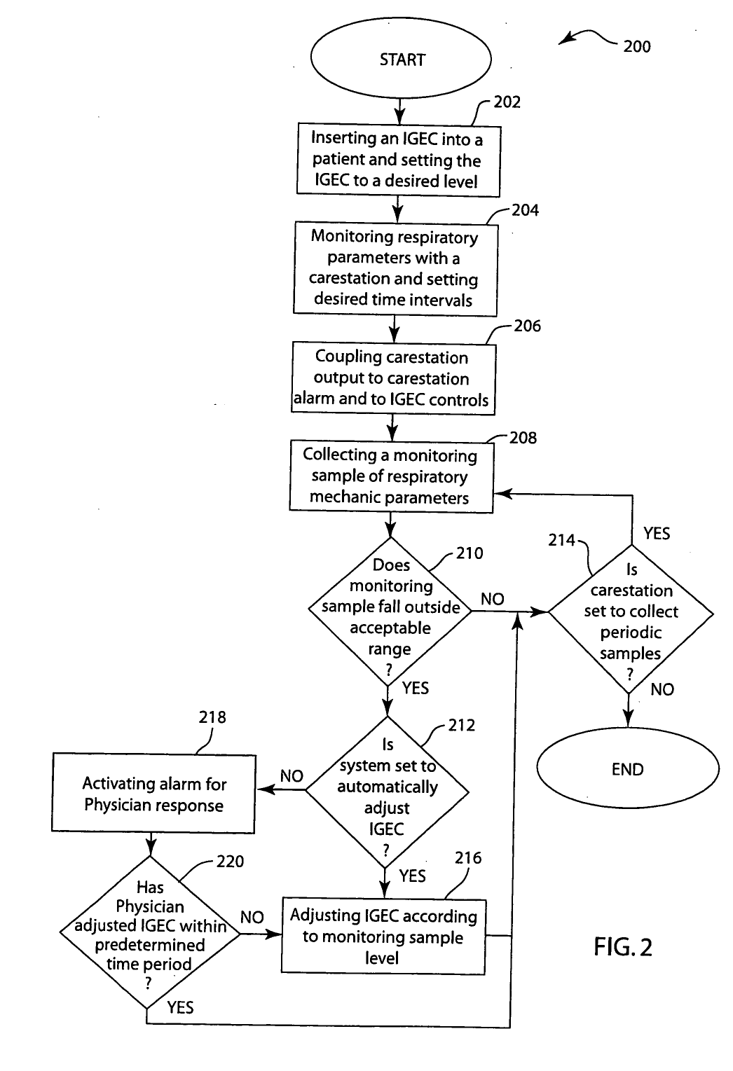

Referring now to FIG. 2, an integration method 200 of the present invention is depicted. In step 202, an IGEC is inserted into a patient and set to a desired oxygenation level. In step 204, the respiratory parameters are monitored with the carestation and desired time levels of sampling these respiratory parameters are set. In step 206, the carestation output is coupled to a carestation alarm, and to the IGEC controls.

Still referring to FIG. 2, in step 208, a monitoring sample of respiratory mechanic parameters are collected by the carestation. In step 210 it is determined whether the monitoring sample falls outside the acceptable range. If the monitoring does not fall outside the acceptable range in step 210, in step 214, it is determined whether the carestation is set to collect periodic samples. If the carestation is so set, then a new monitoring sample is collected in step 208. If the carestation is not set to collect periodic samples, then the integration method ends. Referring back to step 210, if the monitoring sample does fall outside the acceptable range then, in step 212, it is determined whether the system is set to automatically adjust the IGEC. If the system is set to automatically adjust the IGEC, then in step 216, the IGEC is adjusted according to the monitoring sample level, and the integration method 200 continues onto step 214, which is described earlier in this description. Referring back to step 216, the method is also configured such that the ventilator is adjusted in combination with the IGEC.

If the system is not set to automatically adjust the IGEC, then in step 218, an alarm is activated for physician response. In step 220, if the physician has adjusted the IGEC within a predetermined time period, then the integration method 200 continues onto step 214, which is described above. If the physician does not adjust the IGEC within the predetermined time period, then in step 216, the IGEC is automatically adjusted according to the monitoring sample level. In step 220, the physician may also adjust the ventilator in combination with the IGEC.

The respiratory carestation's integration with IGEC delivery allows delivery of respiratory therapy in a more optimized fashion than can be accomplished with prior art systems. Further, when the respiratory carestation includes respiratory mechanics monitoring, automatic assessments of the patient respiratory condition can be accomplished and linked to appropriately aggressive use of the respiratory ventilator.

With the introduction of the integrated respiratory carestations described in this invention, a patient's level of blood oxygenation can be further optimized based on information obtained or generated by the ventilator or respiratory mechanics monitoring system. The respiratory carestation including respiratory mechanics monitoring offers the ability to automatically assess the effectiveness of combined therapy of IGEC and respiratory ventilator (and even control the delivery of such therapy in a closed loop fashion based on the respiratory mechanics monitoring results).

The present invention has been described in terms of specific embodiments incorporating details to facilitate the understanding of the principles of construction and operation of the invention. Such reference herein to specific embodiments and details thereof is not intended to limit the scope of the claims appended hereto. It will be apparent to those skilled in the art that modifications may be made in the embodiment chosen for illustration without departing from the spirit and scope of the invention.

I claim:

1. A method of providing integrated care to a patient with an intravascular gas exchange catheter (IGEC) and a carestation, the method comprising:
a.) collecting a monitoring sample of respiratory parameters with the carestation;
b.) determining whether the monitoring sample is within a predefined acceptable range; and
c.) adjusting the IGEC when the monitoring sample is not within the predetermined range,
wherein adjusting the IGEC controls an amount of oxygen that is added to the bloodstream of the patient and an amount of carbon dioxide removed from the bloodstream of the patient.

2. The method of claim 1, further comprising activating an alarm means when the monitoring sample is not within the predetermined range.

3. The method of claim 2, wherein the adjusting step is effectuated manually by a user.

4. The method of claim 1, further comprising coupling the carestation with the IGEC, and wherein the adjusting step is effectuated automatically when the carestation sends an instruction signal to the IGEC.

5. The method of claim 1 further comprising adjusting a ventilator in combination with the adjusting of the IGEC when the monitoring sample is not within the predetermined range.

6. The method of claim 1, wherein the collecting step is periodically activated when a user sets the carestation to an auto setting.

7. The method of claim 1, further comprising setting the IGEC to a starting level based on a set of patient physiological data.

8. The method of claim 1, further comprising setting the predetermined acceptable range on the carestation based on a set of patient physiological data.

9. The method of claim 1, wherein the IGEC is inserted into the patient through the femoral vein.

10. The method of claim 1, wherein the carestation includes a critical care ventilator, a respiratory monitor and an information management system.

11. The method of claim 10, wherein the carestation is configured to monitor any of the following respiratory parameters:
a.) inspired and expired O2 concentration;
b.) inspired and expired CO2 concentration;
c.) end-tidal CO2 concentration;
d.) CO2 production;
e.) O2 consumption;
f.) metabolics and energy expenditure; and
g.) patient spirometry.

12. A system of providing integrated care to a patient, the system comprising:
a.) a carestation configured to collect a monitoring sample of respiratory parameters, wherein the carestation determines whether the monitoring sample is within a predefined acceptable range; and
b.) an intravascular gas exchange catheter (IGEC) coupled to the carestation and inserted into the bloodstream of the patient, wherein the IGEC is adjustable when the monitoring sample is not within the predetermined range,
wherein adjusting the IGEC controls an amount of oxygen that is added to the bloodstream of the patient and an amount of carbon dioxide removed from the bloodstream of the patient.

13. The system of claim 12, further comprising an alarm means, wherein the alarm means is activated when the monitoring sample is not within the predetermined range.

14. The system of claim 13, wherein the IGEC is adjusted manually by a user.

15. The system of claim 12, wherein the IGEC is adjusted automatically when the carestation sends an instruction signal to the IGEC.

16. The system of claim 12, wherein the carestation includes a critical care ventilator, further wherein the critical care ventilator is adjustable when the monitoring sample is not within the predetermined range.

17. The system of claim 13, wherein the carestation collects the monitoring sample periodically when a user sets the carestation to an auto setting.

18. The system of claim 13, wherein the IGEC is set to a starting level based on a set of patient physiological data.

19. The system of claim 13, wherein the carestation is set to the predetermined acceptable range based on a set of patient physiological data.

20. The system of claim 12, wherein the IGEC is inserted into the patient through the femoral vein.

21. The system of claim 12, wherein the carestation includes a critical care ventilator, a respiratory monitor and an information management system.

22. The system of claim 21, wherein the carestation is configured to monitor any of the following respiratory parameters:
a.) inspired and expired O2 concentration;
b.) inspired and expired CO2 concentration;
c.) end-tidal CO2 concentration;
d.) CO2 production;
e.) O2 consumption;
f.) metabolics and energy expenditure; and
g.) patient spirometry.

23. A method of providing integrated care to a patient with an intravascular gas exchange catheter (IGEC) and a carestation, the method comprising:
a.) coupling the carestation with the IGEC;
b). collecting a monitoring sample of respiratory parameters with the carestation, wherein the carestation includes a critical care ventilator, a respiratory monitor and an information management system;
c.) determining whether the monitoring sample is within a predefined acceptable range;
d.) activating an alarm means when the monitoring sample is not within the predetermined range; and
e.) adjusting the IGEC and the critical care ventilator when the monitoring sample is not within the predetermined range,
wherein adjusting the IGEC controls an amount of oxygen that is added to the bloodstream of the patient and an amount of carbon dioxide removed from the bloodstream of the patient.
